(19) United States
(12) Patent Application Publication
(10) Pub. No.: US 2015/0276496 A1
(43) Pub. Date: Oct. 1, 2015

(54) Thermoelastic Beverage Qualifier

(71) Applicants: Joseph Lawrence Kellogg, Moultonborough, NH (US); Mingming Guo Kellogg, Moultonborough, NH (US)

(72) Inventors: Joseph Lawrence Kellogg, Moultonborough, NH (US); Mingming Guo Kellogg, Moultonborough, NH (US)

(21) Appl. No.: 14/225,283

(22) Filed: Mar. 25, 2014

(51) Int. Cl. G01K 5/48; G01N 33/14

(52) U.S. Cl. CPC G01K 5/483; G01N 33/14

(57) ABSTRACT

A field expedient instrument to be used in the process of qualifying the temperature of hot and cold drinks using thermoelastic materials is presented. The shape recovery properties of thermoelastic materials are used to qualify the temperature of a drink as desirable or undesirable.

### BACKGROUND OF THE INVENTION

There are historical records of Native Americans offering hot drinks to early western explorers in the sixteenth century. Around the time of the American Revolution, the tea trade was booming—culminating in the infamous Boston Tea Party. Today, hot drinks—including coffee, tea, cider, and other drinks—are enjoyed by billions of people worldwide. Since these drinks need to be created at temperatures higher than what can be withstood by the human body, there is a substantial risk of injury when consuming these drinks.

Possibly the most famous instance of an injury from a hot drink is the Liebeck v. McDonalds case, decided on Aug. 18, 1994. Liebeck had purchased a coffee from McDonalds and spilled it on her lap. The ensuing third degree burns required eight days of hospitalization followed by two years of continued medical treatments. The lawsuit that followed ended up receiving a judgment of 2.86 million dollars. While this is an extremely rare case, it shows that people have been aware of the dangers of consuming hot beverages for quite some time.

This brings up the question: what can be done to prevent this type of injury? Some of the solutions are simple, such as labeling the drink as ‘Hot’ to remove any uncertainty. The addition of tight fitting lids with small drinking holes also prevents an excessive quantity of liquid from exiting the container and onto the consumer. However, these safety measures only go so far. When a person takes the first sip of a hot drink, there is a risk that the inside of their mouth can be burned. This may result in anything from discomfort to second degree burns. What is needed is a method of rapidly qualifying the temperature of the drink.

Currently, the best way to quantify the temperature of a hot drink is by using a digital “instant read” thermometer or an infrared thermometer. While infrared thermometers are very fast, however, with price tags starting around $85, they are generally too expensive for the average consumer. Additionally, they are too bulky to store in places such as the armrest of a car. An ‘instant read’ digital thermometer certainly is much smaller and cheaper than an infrared thermometer. However, it has its own problems, the biggest being: 1. it is fragile and 2. despite being called ‘instant read’ it still takes a few seconds to register—a bad thing when drivers are travelling upwards of 50 feet per second. Also, it is well known that it is difficult to interpret a digital readout as opposed to other display types.

This still neglects the fact that the average consumer doesn't know the exact temperature at which they can comfortably begin consuming their beverage. So, while thermometers do an excellent job of quantifying the temperature of a drink, what is really needed is the ability to qualify the temperature of the drink as desirable or undesirable.

To the best knowledge of the inventors, the devices that can be used for qualifying the temperature of a beverage are few and with limited accuracy and usefulness. First, and most accurate, is the device used for determining if a turkey is thoroughly cooked. This usually takes the form of a white plastic body with a red button in the center. This red button is held in place by a spring and a material with a melting temperature equal to the desired temperature of the meat. When this material melts, the spring pushes the red button out, indicating that the meat is cooked. The drawback to these testers is that they are single use, once the button pops, there is no way for the user to put it back.

The second is the color changing pigmented plastic utensils. These are both slow and inaccurate. It can take several seconds for the pigment to change colors. Additionally, many people complain that they change color either way to hot or way too cold, making their usefulness very limited. What is needed is something that is both instant and accurate.

Thermoelastic materials are a class of materials also known as ‘shape memory’ or ‘active’ materials. The thermoelastic phenomenon, on a macroscopic scale, appears to restore a memorized shape after a temperature change. On a molecular level, the thermoelastic phenomenon is made possible by having two or more solid phases with substantially different properties, at relatively low temperatures. Multiple phases were first observed in steel—which has numerous phases, including austenite, pearlite, and martensite among others. The properties of each of these phases are substantially different from each other and can be controlled to get the properties desired in steel.

Now, in thermoelastic materials, the structures are different enough so that, what appears to be a plastic deformation in one phase at the macroscopic level, really hasn't been deformed at all at the crystalline level. At the crystalline level, plastic deformation occurs when the electronic bonds are broken between two sheets of atoms and the crystal slips one or more atomic spacings. However, in thermoelastic materials, when macroscopic deformation occurs, electronic bonds are not broken, however, they merely rotate through a partial atomic spacing. Then, when the temperature is raised above the transition temperature, since the electronic bonds have not been broken, they return to the original form with no permanent deformation. This is why the term thermoelastic is used to describe these materials. While the deformation is not quite 100% recoverable, it is close enough that it may be recovered many thousands of times.

The earliest thermoelastic material discovered was gold-cadmium in 1939. Since then, dozens of alloys have been found to exhibit the thermoelastic phenomena. More recently, thermoelastic polymers have also been developed, allowing for a broadened use of thermoelastic materials.

By varying the chemical make-up and the thermomechanical treatments of a thermoelastic material, the transition temperature can be tuned in rather accurately—often within 1 degree Celsius. Also, the transition temperatures of most thermoelastic materials include the region of 0-100 degrees Celsius, the range of possible temperatures of any drink. This combined capacity of having an accurate transition temperature in the region of temperatures that matter make thermoelastic materials a prime candidate for qualifying the temperature of drinks.

There is such a thing as too much information. If someone is given too much information, it is difficult for them to process it into something useful.

The human eye consists of five major components:
The iris, or the part that controls how much light enters the eye.
The cornea—a protective coating on the outside of the eye.
The lens, which focuses the light onto the retina.
The retina which detects the light.
The optic nerve which transmits the signals from the retina to the brain where it is interpreted into something useful.
The retina detects light using two different types of cells: cones and rods. Cones are capable of detecting color and are used for high resolution vision. The rods are light sensors, they enable vision in very low light, but the resolution of the rods is poor. It makes sense then that the cones and rods are not evenly distributed throughout the eye. The cone cells are concentrated at the macula with the highest concentration being found in the fovea. The remainder of the retina is largely covered with rods. As any engineer would build the eye, the lens focuses most of the light near the fovea. This provides the direct vision that is necessary for life. A human eye with 20/20 vision can distinguish features as small as 1 arc minute using their fovea.

Peripheral vision, while it can only distinguish objects several degrees in resolution, is great at distinguishing motion. While a human is travelling, they use their foveal vision to distinguish things directly in front of them, or things that they deem matter. Meanwhile, the peripheral vision is used to detect unexpected occurrences—motion—at which time the foveal vision is turned to observe more accurately the occurrence. For more information on this, please consult the paper by Thompson.

When operating a motor vehicle, the foveal vision is necessary to discern placement on the road, location of cars, people, animals, trees, etc. The peripheral vision is then used to observe unexpected movement—other cars, animals, people, etc. Both of these are necessary to safe operation of a motor vehicle.

According to the NHSTA, in 2011, distracted driving was an influence in 10% of injury causing crashes. The shape memory properties of thermoelastic materials cause motion when heated or cooled beyond the transition temperature. This makes it so that they could be observed in the peripheral vision—allowing the driver to maintain foveal focus on the road.

### DESCRIPTION OF THE INVENTION

In the invention presented here, a piece of thermoelastic material is used to very rapidly classify the drink as desirable or undesirable. The user inserts the thermoelastic element into the beverage in question. If the temperature of the beverage is above the specified temperature, then the shape change occurs, thereby qualifying the temperature of the beverage as desirable or undesirable.

The proposed embodiment consists of two pieces: 1. the thermoelastic element with a predetermined memory shape which has received thermomechanical treatments and has a chemical make-up that make it suitable to testing drinks in the desired temperature range and 2. a handle allowing the user to insert the thermoelastic element into their beverage without making contact with the liquid.

The thermoelastic element should be constructed in such a way that the heating is rapid so that the user has feedback as to the desirability of the beverage in a very short period of time. The thermoelastic element should also have a chemical makeup that allows it to be reset at ambient temperature. Any additional work required to reset the thermoelastic element significantly reduces the functionality.

### DESCRIPTION OF THE FIGURES

FIG. 1 shows a basic embodiment of the thermoelastic beverage tester. Item 1 is the thermoelastic element used to qualify the beverage temperature. Item 2 is a handle which allows the user to insert the thermoelastic element into their beverage without making contact with said beverage. Item 3 is an optional protective cover, designed to keep the thermoelastic element clean when not in use. Modifications to this design include artistic designs of the handle. The thermoelastic element may also have an artistic or functional design to it.

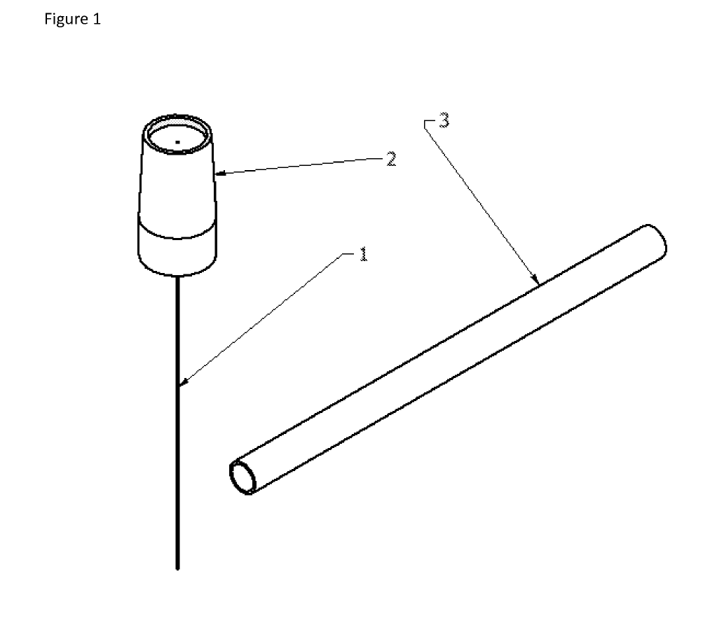

### REFERENCES

Thompson, Benjamin, et. al. Peripheral Vision: Good for Biological Motion, Bad for Signal Noise Segregation? Journal of Vision; Jul. 25, 2007.

1. A device for qualifying the temperature of beverages.

2. Said device utilizing a thermoelastic element to qualify the beverage temperature as desirable or undesirable.
